(19) United States
(12) Patent Application Publication
(10) Pub. No.: US 2003/0092077 A1
(43) Pub. Date: May 15, 2003

(54) Human sperm activator peptides

(76) Inventor: Chodavarapu S. Ramarao, Birmingham, AL (US)

Correspondence Address: BARNES & THORNBURG, 11 South Meridian Street, Indianapolis, IN, 46204, US

(21) Appl. No.: 10/138,089

(22) Filed: May 3, 2002

Related U.S. Application Data
(60) Provisional application No. 60/288,956, filed on May 4, 2001.

(51) Int. Cl. G01N 33/567; C07H 21/04; C12N 9/00; C12P 21/02; C12N 5/06

(52) U.S. Cl. 435/7.21; 435/69.1; 435/183; 435/320.1; 435/325; 530/350; 536/23.2

(57) ABSTRACT

The present invention provides novel peptides that act as chemotactic factors for human sperm. The peptides comprise the amino acid sequence TMGFTAPRFPHY as well as analogs, homologues and derivatives. Also provided is a nucleic acid comprising a nucleotide sequence that encodes a novel sperm chemotactic peptide. Methods for diagnosing infertility caused by defects in sperm-egg interactions are also provided as well as methods for treating infertility wherein the infertility is due to a defect in sperm-egg interactions.

### CROSS REFERENCE TO RELATED APPLICATION

This application claims priority under 35 U.S.C §119(e) to U.S. Provisional Application Serial No. 60/288,956, filed on May 4, 2001.

### SPONSORSHIP

Work on this invention was supported in part by NASA Cooperative Agreement No. NCC8-126. The Government has certain rights in the invention.

### FIELD OF THE INVENTION

The present invention relates generally to human fertilization and more particularly to peptides that activate human sperm.

### BACKGROUND OF THE INVENTION

Approximately 10% of all couples trying to conceive are infertile. Of this number, 10-20% suffer from “unexplained fertility.” While there are a number of procedures available for the diagnosis and treatment of female infertility, there are few procedures to treating infertility caused by defects in the sperm-egg interactions.

Sperm chemotaxis is the response of sperm to a gradient of a stimulus to increase the chances of interaction of the sperm with an egg. Chemotaxis is often accompanied by chemokinesis, an increase in the swimming speed of the sperm in reaction to the stimulus. Sperm chemotaxis is a common phenomenon in marine metazoan including sea urchin, coral, algae and fish. Until recently, there was speculation but little evidence that sperm chemotaxis occurred in mammals, especially in humans. A factor has been partially isolated, but not identified, in human follicular fluid that increases sperm chemotaxis. U.S. Pat. No. 5,849,713; Felterolf, P. M. et al., Human Reproduction 9,1505-1511 (1994). This factor is thought to be a peptide having a molecular weight of at least 1.0 kD.

It would thus be desirable to isolate and identify the human factor responsible sperm chemotaxis. Currently, a factor is derived in impure form from human follicular fluid in limited amounts. Determination of the sequence of the human factor would allow for large scale production resulting in availability for treatment of a wide population for infertility.

### SUMMARY OF THE INVENTION

The present invention provides novel peptides that act as chemotactic factors for human sperm. The peptides comprises the amino acid sequence TMGFTAPRFPHY (SEQ. ID. NO. 1) as well as analogs, homologues and derivatives. Also provided is a nucleic acid molecule comprising a nucleotide sequence that encodes a novel sperm chemotactic peptide (SEQ. ID. NO. 2).

The present invention further provides methods for diagnosing infertility caused by defects in sperm-egg interactions. Methods are also provided for treating infertility wherein the infertility is due to a defect in sperm-egg interactions. For example, the sperm chemotactic factor of the present invention may be used to increase fertilization both in vivo and in vitro.

Additional objects, advantages, and features of the present invention will become apparent form the following description, taken in conjunction with the accompanying drawings.

### BRIEF DESCRIPTION OF THE DRAWINGS

The various advantages of the present invention will become apparent to one skilled in the art by reading the following specification and by referencing the following drawings in which:

FIG. 1 is a photomicrograph showing the binding of fluorescent-labeled sperm chemotactic peptide to sperm; and

FIG. 2 is a bar graph showing the effect of increasing concentrations of sperm chemostactic peptide on the binding of fluorescent-labeled peptide to sperm.

### DETAILED DESCRIPTION OF THE INVENTION

The present invention provides a human sperm activator peptide (Husap) that binds to human sperm and acts as a human sperm chemotactic factor. The sequence of the peptide is NH₂-Thr-Met-Gly-Phe-Thr-Ala-Pro-Arg-Phe-Pro-His-Tyr-COOH (or TMGFTAPRFPHY) (SEQ. ID. NO. 1).

The peptide of the present invention was identified using phage display technology. Sperm were bound onto the well surfaces of a microtiter plate. Phage were then added to the wells and those that specifically bound were isolated and amplified. DNA was isolated from the phage and the peptide sequences expressed on the phage surface responsible for binding to the sperm were identified. The Husap peptide, TMGFTAPRFPHY (SEQ. ID. NO. 1) was then further tested for its chemotactic properties.

The present invention also provides other variants and derivatized species of the Husap peptide that can be used in the present invention. Variants will include analogs, homologs, and derivatives that retain the ability to bind to sperm and induce chemotaxis. Fragments of the peptide retaining chemotactic activity are also contemplated.

Analysis of the Husap peptide can include, but are not limited to peptides in which conservative amino acid substitutions are made. By “conservative substitutions” it is meant replacing an amino acid residue with another that is biologically, chemically or spatially similar, e.g., one hydrophobic residue for another, or one polar residue for another. Such conservative substitutions are well known to the skilled artisan. For example, but not limited to, Gly and Ala, Val and Ile, Asp and Glu, Asn and Gin, Ser and Thr, Lys and Arg and Phe and Tyr are pairs of amino acids that can replace each other. Preferably, the variant sequence will differ less than 50% from SEQ. ID. NO. 1, except where additional amino acids may be added at either terminus. More preferably, the variant sequence will differ less than 85% at the amino terminus portion of the peptide. A non-limiting example of a variant sequence is TMVFTNPRISQY (SEQ. ID. NO.3).

Nucleic acid molecules having nucleotide sequences that encodes for the Husap peptide is also provided. Preferably, the nucleic acid has the nucleotide sequence 5′actatggsttttacggctccgcggtttccgcattta-3′ (SEQ. ID. NO. 2) or its complementary sequence. Additional sequences can be derived by the skilled artisan from the known amino acid sequence of SEQ. ID. NO. 1 and based on the known degeneracy of the genetic code. Also provided are nucleic acids having a nucleotide sequence that hybridizes under stringent conditions the nucleotide sequence of SEQ. ID. NO. 2 or its complementary sequence.

The nucleic acids of the present invention may be used to express the Husap protein both in vitro and in vivo. The nucleic acid is placed in the appropriate vector or other suitable construct and a host cell is transformed with the vector or construct. Non-limiting examples of cells that can be used to express the Husap protein are bacteria, yeast and mammalian cells. Preferably, the Husap peptide is expressed in E. coli or human ovarian cells.

Among the utilities for sperm-attracting compositions comprising the peptides of the present invention is a process of assaying the fitness of a population of sperm for fertilization. The ability of the human sperm sample to be attracted to peptides capable of attracting sperm, in any of their various forms or purities, is directly related to the ability of that sperm sample to cause fertilization. In particular, the assay may be done on a number of sperm populations. The population with the greater ability to be attracted to the sperm-attracting composition is considered to be the population with the greatest fitness for fertilization. The method of the present invention includes contacting a solution containing at least one of the chemotactic peptides of the present invention with a semen sample containing sperm and detecting chetaxis of the sperm in the semen sample. Alternatively, the amount of chemotaxis can be quantified and compared to a reference value. By way of non-limiting example, a reference value can be determined by the disclosed methods using a sperm sample known to be viable for fertilization.

A related utility for the chemotactic factor(s) of the invention is sperm improvement without physical separation of the cells. Preincubation of the sperm with the chemotactic peptides may be used for increasing the fraction of capacitated sperm as pre-treatment for assisted in vivo and in vitro fertilization.

Another utility for the chemotactic peptides involves actually separating or selecting from a given sperm sample only those sperm cells which are most greatly attracted to the chemotactic peptides. Any method of selecting the sperm which are most responsive to the peptides may be used. For example, two wells connected by a tube can be provided, one well containing spermatozoa and the other containing the sperm-attracting preparation. A gradient of such factor would be formed along the tube and responsive spermatozoa would follow the gradient and accumulate in the other well. Another possibility is to make a gradient in a viscous medium by a gradient maker. Yet another possibility is to use a semi-solid medium in which spermatozoa are added at one end and a polymer soaked with the sperm-attracting composition is added at the other end. A gradient of the factor in this case will be made by sustained release from the polymer. Those of ordinary skill in the art will understand that many other chambers and vessels can be suggested, but that the principle would be the same in all cases. That portion of the sperm population that moves most rapidly in the direction of increasing concentration of factor is that which is most fit for fertilization processes. The capacitated sperm that have been separated out can then be used in either in vitro or in vivo fertilization.

Another aspect of the present invention is the prediction of the relative fitness of an egg for fertilization. It has been found that eggs from follicles having the greatest amount of the chemotactic factors of the present invention in their follicular fluid are most likely to have positive fertilization. Thus, for example, the relative fitness of eggs for in vitro fertilization can be determined by taking a sample of follicular fluid from the same follicle as the one in which the egg was found, and measuring the amount of the chemotactic peptides of the present invention in the fluid. The probability of fitness for fertilizability of a plurality of eggs tested in this manner can be assigned based on the relative amount of chemotactic peptides present. By way of a non-limiting example, the amount of the chemotactic peptide can be determined by immunochemical methods such as ELISA and radioimmunoassay using antibodies raised against the chemotactic peptides. A greater probability of fitness for fertilizability is assigned to an egg from a follicle that produces the fluid with higher concentrations of the chemotactic peptides. The assay may also be carried out in other body fluids, such as serum or urine, to which the chemotactic factors have been passed from the bursting follicle. Concentration of the peptides below a threshold concentration would indicate low probability of fertilization. The threshold concentration may be obtained by the skilled artisan by determining the peptide concentration by the same method in an egg known to be fit for fertilization.

The peptides of the invention and compositions containing them may be also useful as contraceptive agents in intrauterine devices or in contraceptive foams and gels, since they mask the sperm receptors and prevent them from reaching the egg. Another approach would be to use blockers of the sperm chemotaxis receptors. Alternatively, antibodies against chemotactic peptides of the present invention may also be used as contraceptive agents. Such antibodies would bind chemotactic peptides produced in vivo resulting in unfertilization of the egg.

The peptides of the present invention may also be used to isolate the receptor(s) on the sperm that bind the peptides. Identification of the receptors would allow for the development of compounds that block binding of the chemotactic peptides to the sperm. Such compounds could then be used as contraceptive agents.

As referred to herein, the term “nucleic acid” is intended to mean natural and/or synthetic linear, circular and sequential arrays of nucleotides and nucleosides, e.g., cDNA, genomic DNA (gDNA), mRNA, and RNA, oligonucleotides, oligonucleosides, and derivatives thereof. The term “encoding” is intended to mean that the subject nucleic acid may be transcribed and translated into either the desired polypeptide or the subject protein in an appropriate expression system, e.g., when the subject nucleic acid is linked to appropriate control sequences such as promoter and enhancer elements in a suitable vector (e.g., an expression vector) and when the vector is introduced into an appropriate system or cell.

As referred to herein, the term “capable of hybridizing under high stringency conditions” means annealing a strand of DNA complementary to the DNA of interest under highly stringent conditions. Likewise, “capable of hybridizing under low stringency conditions” refers to annealing a strand of DNA complementary to the DNA of interest under low stringency conditions. In the present invention, hybridizing under either high or low stringency conditions would involve hybridizing a nucleic acid sequence (e.g., the complementary sequence to SEQ. ID No: 2 or portion thereof), with a second target nucleic acid sequence. “High stringency conditions” for the annealing process may involve, for example, high temperature and/or lower salt content, which disfavor hydrogen bonding contacts among mismatched base pairs. “Low stringency conditions” would involve lower temperature, and/or higher salt concentration than that of high stringency conditions. Such conditions allow for two DNA strands to anneal if substantial, as is the case among DNA strands that code for the same protein but differ in sequence due to the degeneracy of the genetic code. Appropriate stringency conditions which promote DNA hybridization, for example, 6× SSC at about 45° C., followed by a wash of 2× SSC at 50° C., are known to those skilled in the art or can be found in Current Protocols in Molecular Biology, John Wiley & Sons, NY (1989), 6.31-6.3.6. For example, the salt concentration in the wash step can be selected from a low stringency of about 2× SSC at 50 C, to a high stringency of about 0.2× SSC at 50° C. In addition, the temperature in the wash step can be increased from low stringency at room temperature, about 22° C., to high stringency conditions, at about 65° C. Other stringency parameters are described in Maniatis, T., et al., Molecular Cloning: A Laboratory Manual, Cold Spring Harbor Laboratory Press, Cold Spring N.Y., (1952), at pp. 387-389; see also Sambrook J. et al., Molecular Cloning: A Laboratory Manual, Second Edition, Volume 2, Cold Spring Harbor Laboratory Press, Cold Spring, N.Y. at pp. 8.46-8.47 (1989).

The foregoing and other aspects of the invention may be better understood in connection with the following examples, which are presented for purposes of illustration and not by way of limitation.

### EXAMPLE 1

Identification of Peptide Sequences

A cryopreserved sample of human sperm was obtained from Fairfax Sperm Bank, Fairfax, Va. The sperm sample was thawed at room temperature and swim up sperm were collected in Hams-F10 buffer (Life Technologies). Hams buffer (1 ml) was layered over the sperm solution and incubated at 37° C. for 1 h after which 800 p1 of the upper layer was removed. The sperm were isolated from the upper layer by low speed centrifugation and washed twice with Hams F10 buffer. The spermatozoan pellet was then re-suspended in Hams F10 buffer and counted in a hemocytometer. Sperm cells (4×10⁶) were resuspended in 100 ml of Hams buffer and coated onto the well surfaces of a micro-titer tissue culture plate. The sperm were allowed to bind by incubation at 8° C. overnight on a rocking platform, after which any unbound sperm were removed. Non-specific binding of phage samples with the sperm was blocked by incubating the wells with 0.25 ml of blocking solution composed of 0.1M NaHCO₃ buffer (pH-8.0) consisting of 5 mg/mL BSA and 0.02% NaN₃ for 1 hr. at 8° C. The blocking solution was then removed by aspiration and the wells rapidly washed 6 times with TBST (Tris Buffered Saline containing 0.1% Tween-20). Phage diluted in 100 μl of TBS-T to a concentration of 4×10¹¹ were incubated in the sperm-coated wells at room temperature for 1 h. At the end of the incubation period, the unbound phage were removed and the wells washed 10 times with TBS-T. The bound phage were eluted from the wells with 100 p1 of 0.2M Glycine-HCI (pH 2.2) buffer, and immediately neutralized with 15 μl of 1M Tris-HCL (pH 9. 1).

The phage pools expressing sequences that specifically bound to the sperm surface were then amplified in an F+ strain of E. Coli (ER2738, New England Biolabs). The amplified phage expressing the same sequences were then enriched by two more rounds of panning. After the third panning, the eluted phage were plated on LB-Tetracycline plates containing IPTG and X-Gal. Fifteen individual blue plaques were picked and phage DNA prepared from them. The DNA was sequenced and the corresponding peptide sequences expressed on the phage surface responsible for binding to the sperm were deduced.

Peptide sequences identified by phage display screening of human sperm are as follows:

| | | |
|---|---|---|
| sperm 1 pro | HS HMRLSQWPLLKP GGG | (SEQ.ID.NO.4) |
| sperm 2 pro | HS HLPTSVTHFYVS GGG | (SEQ.ID.NO.5) |
| sperm 3 pro | HS TMGFTAPRFPHY GGG | (SEQ.ID.NO.6) |
| sperm 4 pro | HS WPHNWWPHFKVK GGG | (SEQ.ID.NO.7) |
| sperm 5 pro | HS SAHGTSTGVPWP GGG | (SEQ.ID.NO.8) |
| sperm 6 pro | HS TMGFTAPRFPHY GGG | (SEQ.ID.NO.6) |
| sperm 8 pro | HS AHRHPISFLSTL GGG | (SEQ.ID.NO.9) |
| sperm 9 pro | HS TMGFTAPRFPHY GGG | (SEQ.ID.NO.6) |
| sperm 10 pro | HS TMGFTAPRFPHY GGG | (SEQ.ID.NO.6) |
| sperm 11 pro | HS WPHNWWPHFKVK GGG | (SEQ.ID.NO.7) |
| sperm 12 pro | HS STTKIPNPVHWR GGG | (SEQ.ID.NO.10) |
| sperm 13 pro | HS TMGFTAPRFPHY GGG | (SEQ.ID.NO.6) |
| sperm 14 pro | HS SNIRLSNSPMWT GGG | (SEQ.ID.NO.11) |
| sperm 15 pro | HS TMGFTAPRFPHY GGG | (SEQ.ID.NO.6) |
| sperm 16 pro | HS ATWSHHLSSAGL GGG | (SEQ.ID.NO.12) |
| sperm 17 pro | HS TPFLPNVGTFSR GGG | (SEQ.ID.NO.13) |
| sperm 18 pro | HS AFTHEAPKRLDS GGG | (SEQ.ID.NO.14) |
| sperm 19 pro | HS ANCHHIRFLCNL GGG | (SEQ.ID.NO.15) |
| sperm 21 pro | HS TMVFTNPRISQY GGG | (SEQ.ID.NO.16) |
| sperm 22 pro | HS TMGFTAPRFPHY GGG | (SEQ.ID.NO.6) |
| sperm 26 pro | HS GPRAAEIQSGVF GGG | (SEQ.ID.NO.17) |
| sperm 28 pro | HS SNFKTPLPLTQS GGG | (SEQ.ID.NO.18) |
| sperm 29 pro | HS QAHLEKTAVPRM GGG | (SEQ.ID.NO.19) |

### EXAMPLE 2

Effect of Husap on Sperm Chemotaxis

Preparation of sperm: One tube (0.5 ml) of a sperm sample was thawed at room temperature for 15-20 mm. As soon as the sperm was thawed, 0.5 ml of Hams-F10 medium was added, and the sample transferred to a 5 ml culture tube. The sperm was diluted to 4 ml with Hams-F10 medium, mixed well, and centrifuged at 2000 rpm for 5 mm. The supernatant was decanted and the cells resuspended in 4 ml of fresh Hams-F 10 medium. The centrifugation was repeated and the clean sperm pellet resuspended in 1 ml Hams-F10 medium. A 50 μl sample aliquot was used per each assay condition.

Assay: The chambers of the assay apparatus were assembled with 8.iM membranes pre-wetted with Hams-F10 medium. A sample of phage containing a specific peptide sequence or a control peptide sequence was diluted with Hams-F10 medium to give a 100 ml phage containing solution which was placed in the bottom chamber of the apparatus. A sperm sample having at least 1×10⁶ sperm was added to the upper chamber. After incubation 37° C. for 1 hr, the sample in the bottom chamber was collected into microfuge tubes and centrifuged at 6000 rpm for 5 mm. The supernatant was removed and the pellet resuspended in suitable volume to be counted in a hemocytometer.

Results: Various concentrations of phage containing the Husap peptide (TMGFTAPRFPHY; SEQ. ID. NO. 1), phage containing a control sequence (KSLSRHDHIHHH; SEQ. ID. NO. 20) or buffer without phage were used in the assays. Sperm counts 2-15 fold greater in the well containing the Husap peptide displaying phage were consistently obtained when compared to control phage or buffer-alone chambers. For example, in one experiment done in triplicate, the sperm count in the lower chamber was 76×10⁴ for the Husap peptide compared to 5×10⁴ for the control peptide and 21×10⁴ for buffer alone.

The effect of varying concentrations of the Husap peptide on chemotaxis of human sperm was also determined. Sperm at a concentration of 2.8×10⁶ were added to the upper chamber. The peptide concentration in the lower chamber was varied from 0 μl to 100±1 of peptide. Sperm were separated from the lower chamber by a 5 micron membrane filter. The chambers were incubated at 37° C. for 1 h. At the end of the incubation, sperm were collected from the lower chamber and counted in a hemocytometer. The experiment was performed several times with varying peptide concentrations and with 5 and 8 micron membranes.

There was a peptide concentration effect on sperm chemotaxis as shown in Table 1.

TABLE 1

| HusaP Concentration (uM) | Sperm Count in Lower Chamber |
|---|---|
| 0 | 0 |
| 10 | 6 × 10⁴ |
| 20 | 12 × 10⁴ |
| 100 | 52 × 10⁴ |

### EXAMPLE 3

Determination of Binding of Husap to Sperm

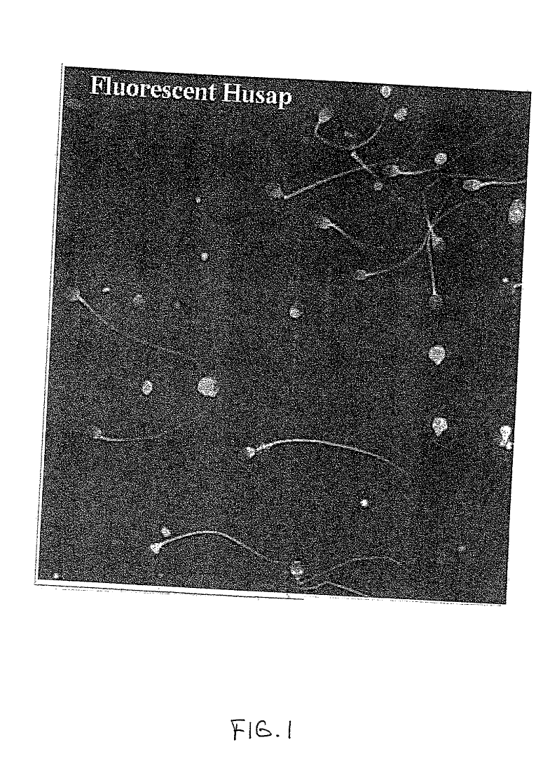

Sperm were incubated with the Husap peptide having a fluorescein fluorescent label to determine the extent of the binding of the peptide to the sperm. It was found that while the peptide bound to the sperm (FIG. 1), binding was not 100%. A comparison of the sperm binding the fluorescent peptide and those stained with Hoechst Dye 33256 showed that only about 60% of the sperm bound the peptide.

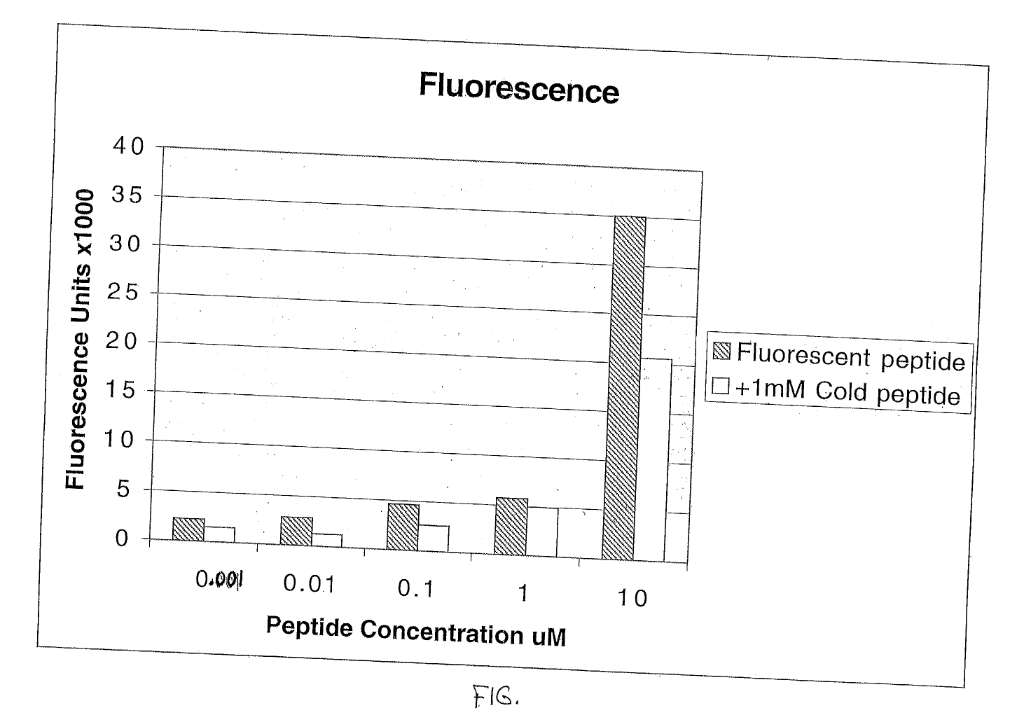

Further, the peptide concentration was shown to have an effect on the binding of the peptide to the sperm (FIG. 2). As the peptide concentration increased, the fluorescence of the sperm sample also increased. Binding of the fluorescent peptide decreased upon addition of unlabeled peptide, suggesting that the binding is specific.

The foregoing discussion discloses and describes merely exemplary embodiments of the present invention. One skilled in the art will readily recognize from such discussion, and from the accompanying drawings and claims, that various changes, modifications and variations can be made therein without departing from the spirit and scope of the invention as defined in the following claims.

All patents and other publications cited herein are expressly incorporated by reference.

1. A chemotactic peptide comprising the amino acid sequence of SEQ. ID. NO. 1 or analogs thereof.

2. The chemotactic peptide of claim 1 wherein the amino acid sequence of the peptide is at least 85% homologous to SEQ. ID. NO. 1.

3. An isolated nucleic acid molecule comprising a nucleotide sequence encoding the chemotactic peptide of claim 1.

4. A vector comprising the nucleic acid of claim 3.

5. A cell comprising the vector of claim 4.

6. The cell of claim 5, wherein the cell is a human ovarian cell or an E. coli cell.

7. The isolated nucleic acid of claim 3 wherein the nucleotide sequence is capable of hybridizing to SEQ. ID. NO. 2.

8. A method for detecting male infertility from a semen sample comprising the steps of:
(a) contacting a solution of the chemotactic peptide of claim 1 with a semen sample;
(b) detecting chemotaxis of sperm in the sperm sample; and
(c) comparing the amount of chemotaxis with a reference value wherein the reference value is obtained from a known fertile sample.

9. A method for increasing in vitro or in vivo fertilization of a mammalian egg cell comprising the steps of:
(a) contacting a mammalian sperm sample with the chemotactic peptide of claim 1; and
(b) further contacting the sperm sample of step (a) with a mammalian egg cell.

10. The method of claim 9 further comprising the step of selecting from the sperm sample in step (a) sperm cells which respond to the chemotactic peptide and contacting the responding sperm cells with the mammalian egg in step (b).

11. A method for determining mammalian female infertility comprising the steps of:
(a) isolating follicular fluid and an egg cell from a mammalian follicle;
(b) detecting the presence of the chemotactic peptide of claim 1 in the follicular fluid; and
(c) comparing the amounts of chemotactic peptide with a reference value.

12. A contraceptive agent comprising the chemotactic peptide of claim 1.

13. An isolated nucleic acid molecule encoding a chemotactic peptide comprising a nucleotide sequence of SEQ. ID. NO. 2, or the complementary sequence of SEQ. ID. NO. 2.

14. A vector comprising the nucleic acid molecule of claim 13.

15. A cell comprising the vector of claim 14.

16. The cell of claim 15 wherein the cell is a human ovarian cell or an E. coli cell.

17. A method for detecting male infertility from a semen sample comprising the steps of:
(a) contacting a solution of chemotactic peptide comprising the amino acid sequence of SEQ. ID. NO. 1 with a semen sample;
(b) detecting chemotaxis of sperm in the sperm sample; and
(c) comparing the amount of chemotaxis with a reference value.

18. A method for increasing in vitro or in vivo fertilization of a mammalian egg cell comprising the steps of:
(a) contacting a mammalian sperm sample with a chemotactic peptide comprising the amino acid sequence of SEQ. ID. NO. 1; and
(b) further contacting the sperm sample of step (a) with a mammalian egg cell.

19. The method of claim 18 further comprising the step of selecting from the sperm sample in step (a) sperm cells which respond to the chemotactic peptide and contacting the responding sperm cells with the mammalian egg in step (b).


SEQUENCE LISTING (20 sequences)

SEQ ID NO: 1 - protein, 12 aa, Homo sapiens
TMGFTAPRFPHY

SEQ ID NO: 2 - DNA, 36 bp, Homo sapiens
actatggsttttacggctccgcggtttccgcattat

SEQ ID NO: 3 - protein, 12 aa, Homo sapiens
TMVFTNPRISQY

SEQ ID NO: 4 - protein, 17 aa, Homo sapiens
HSHMRLSQWPLLKPGGG

SEQ ID NO: 5 - protein, 17 aa, Homo sapiens
HSHLPTSVTHFYVSGGG

SEQ ID NO: 6 - protein, 17 aa, Homo sapiens
HSTMGFTAPRFPHYGGG

SEQ ID NO: 7 - protein, 17 aa, Homo sapiens
HSWPHNWWPHFKVKGGG

SEQ ID NO: 8 - protein, 17 aa, Homo sapiens
HSSAHGTSTGVPWPGGG

SEQ ID NO: 9 - protein, 17 aa, Homo sapiens
HSAHRHPISFLSTLGGG

SEQ ID NO: 10 - protein, 17 aa, Homo sapiens
HSSTTKIPNPVHWRGGG

SEQ ID NO: 11 - protein, 17 aa, Homo sapiens
HSSNIRLSNSPMWTGGG

SEQ ID NO: 12 - protein, 17 aa, Homo sapiens
HSATWSHHLSSAGLGGG

SEQ ID NO: 13 - protein, 17 aa, Homo sapiens
HSTPFLPNVGTFSRGGG

SEQ ID NO: 14 - protein, 17 aa, Homo sapiens
HSAFTHEAPKRLDSGGG

SEQ ID NO: 15 - protein, 17 aa, Homo sapiens
HSANCHHIRFLCNLGGG

SEQ ID NO: 16 - protein, 17 aa, Homo sapiens
HSTMVFTNPRISQYGGG

SEQ ID NO: 17 - protein, 17 aa, Homo sapiens
HSGPRAAEIQSGVFGGG

SEQ ID NO: 18 - protein, 17 aa, Homo sapiens
HSSNFKTPLPLTQSGGG

SEQ ID NO: 19 - protein, 17 aa, Homo sapiens
HSQAHLEKTAVPRMGGG

SEQ ID NO: 20 - protein, 12 aa, Homo sapiens
KSLSRHDHIHHH